(19) United States
(12) Patent Application Publication
(10) Pub. No.: US 2023/0065702 A1
(43) Pub. Date: Mar. 2, 2023

(54) SINGLE MONOLITHIC PIECE PIVC-INTEGRATED HEMOLYSIS-REDUCTION ACCESSORIES FOR DIRECT BLOOD DRAW

(71) Applicant: CareFusion 303, Inc., San Diego, CA (US)

(72) Inventor: Abin AUSTIN, Thrissur (IN)

(21) Appl. No.: 17/895,887

(22) Filed: Aug. 25, 2022

Related U.S. Application Data
(60) Provisional application No. 63/237,946, filed on Aug. 27, 2021.

(51) Int. Cl. A61M 39/12; A61M 25/00

(52) U.S. Cl. CPC A61M 39/12; A61M 25/0097; A61M 2039/1083

(57) ABSTRACT

A flow restriction device may include a male luer connector portion, a female luer connector portion disposed proximally to the male luer connector portion, and a body portion extending between the male and female luer portions, and integrally formed with the male and female luer portions to form a single monolithic piece. The male luer connector portion may have an internal surface defining a lumen thereof, and the female luer connector portion may have an internal surface defining a lumen fluidly connected to the lumen of the male luer connector portion. The body portion may have a recess extending longitudinally therethrough and fluidly communicated with the lumens of the male luer portion and the female luer portion. The recess may define a micro-channel along which fluid flows from the male luer connector portion into the fluid collection device via the female luer connector portion.

### CROSS-REFERENCE TO RELATED APPLICATIONS

This application claims the benefit of U.S. Provisional Application No. 63/237,946, titled “SINGLE MONOLITHIC PIECE PIVC-INTEGRATED HEMOLYSIS-REDUCTION ACCESSORIES FOR DIRECT BLOOD DRAW,” filed Aug. 27, 2021, the disclosure of which is incorporated herein by reference in its entirety.

### TECHNICAL FIELD

The present disclosure generally relates to blood draw and administration of parenteral fluids to a patient, and particularly to systems and methods to reduce hemolysis in PIVC blood draw using a single monolithic piece flow restriction device.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is an over-the-needle peripheral intravenous (“IV”) catheter (PIVC). As its name implies, the over-the-needle catheter may be mounted over an introducer needle having a sharp distal tip. A catheter assembly may include a catheter hub, the catheter extending distally from the catheter hub, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

For blood withdrawal or collecting a blood sample from a patient, a blood collection container may be used. The blood collection container may include a syringe. Alternatively, the blood collection container may include a test tube with a rubber stopper at one end. In some instances, the test tube has had all or a portion of air removed from the test tube so pressure within the test tube is lower than ambient pressure. Such a blood collection container is often referred to as an internal vacuum or a vacuum tube. A commonly used blood collection container is a VACUTAINER® blood collection tube, available from Becton Dickinson & Company.

The blood collection container may be coupled to the catheter. When the blood collection container is coupled to the catheter, a pressure in the vein is higher than a pressure in the blood collection container, which pushes blood into the blood collection container, thus filling the blood collection container with blood. A vacuum within the blood collection container decreases as the blood collection container fills, until the pressure in the blood collection container equalizes with the pressure in the vein, and the flow of blood stops.

Unfortunately, as blood is drawn into the blood collection container, red blood cells are in a high shear stress state and susceptible to hemolysis due to a high initial pressure differential between the vein and the blood collection container. Hemolysis may result in rejection and discard of a blood sample. The high initial pressure differential can also result in catheter tip collapse, vein collapse, or other complications that prevent or restrict blood from filling the blood collection container. Furthermore, blood spillage during and/or after blood draw commonly occurs.

The description provided in the background section should not be assumed to be prior art merely because it is mentioned in or associated with the background section. The background section may include information that describes one or more aspects of the subject technology.

### SUMMARY

The present disclosure provides a flow restriction device, comprising: a male luer connector portion configured to couple to a catheter assembly, the male luer connector portion comprising an internal surface defining a lumen thereof; a female luer connector portion disposed proximally to the male luer connector portion and configured to couple to a fluid collection device, the female luer connector portion comprising an internal surface defining a lumen fluidly connected to the lumen of the male luer connector portion; and a body portion extending between the male luer portion and the female luer portion, and integrally formed with the male luer portion and the female luer portion to form a single monolithic piece, the body portion comprising a recess extending longitudinally therethrough and fluidly communicated with the lumens of the male luer portion and the female luer portion, wherein the recess defines a micro-channel along which a fluid flows from the male luer connector portion into the fluid collection device via the female luer connector portion.

In some instances, the present disclosure provides a method of manufacturing a single monolithic piece flow restriction device configured to limit hemolysis during the drawing of blood from a patient, the method comprising: providing first and second mold parts, each of the first and second mold parts comprising an imprint recessed therein, the imprint comprising a male luer connector imprint portion at a distal end portion of the first mold part, a female luer connector imprint portion at a proximal end portion thereof, a micro-channel imprint portion extending longitudinally between the male luer connector imprint portion and the female luer connector imprint portion, and a reinforcing rib imprint portion surrounding the micro-channel imprint portion; inserting a male luer connector mold part into the male luer connector imprint portion; inserting a female luer connector mold part into the female luer connector imprint portion, the female luer connector mold part comprising a micro-channel mold part extending from a distal end of the female luer connector mold part, wherein the micro-channel mold part is inserted into the micro-channel imprint portion; coupling the first and second mold parts to form a mold assembly; injecting a molten mold material into the mold assembly; and cooling the mold assembly.

In some aspects, the present disclosure provides a flow restriction device, comprising: first and second connector halves fused together to form a single connector, the single connector comprising: a male luer connector portion configured to couple to a catheter assembly, the male luer connector portion comprising an internal surface defining a lumen thereof; a female luer connector portion disposed proximally to the male luer connector portion and configured to couple to a fluid collection device, the female luer connector portion comprising an internal surface defining a lumen fluidly connected to the lumen of the male luer connector portion; and a body portion extending between the male luer portion and the female luer portion, and integrally formed with the male luer portion and the female luer portion to form a single monolithic piece, the body portion comprising at least one recess extending longitudinally therethrough and fluidly communicated with the lumens of the male luer portion and the female luer portion, wherein the at least one recess defines a micro-channel along which a fluid flows from the male luer connector portion into the fluid collection device via the female luer connector portion.

In some instances, the present disclosure provides a peripheral intravenous catheter assembly configured to limit hemolysis during the drawing of blood from a patient, comprising: a flow restriction device, comprising, a male luer connector portion configured to couple to a catheter assembly, the male luer connector portion comprising an internal surface defining a lumen thereof; a female luer connector portion disposed proximally to the male luer connector portion and configured to couple to a fluid collection device, the female luer connector portion comprising an internal surface defining a lumen fluidly connected to the lumen of the male luer connector portion; and a body portion extending between the male luer portion and the female luer portion, and integrally formed with the male luer portion and the female luer portion to form a single monolithic piece, the body portion comprising a recess extending longitudinally therethrough and fluidly communicated with the lumens of the male luer portion and the female luer portion, wherein the recess defines a micro-channel, the micro-channel configured to limit hemolysis of blood as the blood is drawn from a patient from the male luer connector portion into the fluid collection device via the female luer connector portion; a catheter hub having a proximal end and a distal end; and a fluid connector that fluidly couples the catheter hub with the flow restriction device.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1 illustrates a vascular access device including a peripheral intravenous catheter (PIVC) assembly that includes a flow restriction device, in accordance with some embodiments of the present disclosure.

FIG. 2A illustrates a perspective view of the flow restriction device, in accordance with some embodiments of the present disclosure.

FIG. 2B illustrates a top view of the flow restriction device of FIG. 2A, in accordance with some embodiments of the present disclosure.

FIG. 2C illustrates a cross-sectional view of the flow restriction device of FIG. 2B along line 2C-2C, in accordance with some embodiments of the present disclosure.

FIG. 2D illustrates a side view of the flow restriction device of FIG. 2A, in accordance with some embodiments of the present disclosure.

FIG. 2E illustrates a cross-sectional view of the flow restriction device of FIG. 2D along the line 2E-2E, in accordance with some embodiments of the present disclosure.

FIG. 3A illustrates perspective view of a plurality of mold parts for forming a flow restriction device, in accordance with some embodiments of the present disclosure.

FIG. 3B illustrates an assembled mold of the flow restriction device without a top mold of the flow restriction device, in accordance with some embodiments of the present disclosure.

FIG. 4 illustrates a cross-sectional view of first and second connector halves of a flow restriction device, in accordance with some embodiments of the present disclosure.

FIG. 5A illustrates a perspective view of a flow restriction device, in accordance with some embodiments of the present disclosure.

FIG. 5B illustrates a cross-sectional view of first and second halves of the flow restriction device of FIG. 5A, in accordance with some embodiments of the present disclosure.

FIG. 6 illustrates a cross-sectional view of first and second connector halves of a flow restriction device, in accordance with some embodiments of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Blood draw via a vascular access device has drawn increasing attention attributed to minimized needle sticks and improved operation efficiency as compared with traditional blood draw methods with venipuncture. Current blood draw using a peripheral intravenous catheter (PIVC) has seen some challenges, one of the most critical is hemolysis related blood quality. In particular, with currently existing PIVC products in the market, along with the standard connection (such as a short extension set and a needleless connector), and blood collection devices (such as a Vacutainer), the shear stress exerted onto blood cells tends to be on the verge of hemolyzing.

Various embodiments of the present disclosure are directed to providing systems and methods to address hemolysis in PIVC blood draw with a hemolysis reduction accessory (also referred to herein as a flow restriction device) which is pre-attached to the PIVC and serves as a flow restrictor to reduce risk of hemolysis. The hemolysis-reduction accessory is advantageously compatible with PIVC placement and does not necessitate change to any of the existing operations. The hemolysis-reduction accessory of the various embodiments described herein is potentially applicable to a wide variety of PIVC products, and compatible with existing blood collection devices and infusion disposables.

Various embodiments of the present disclosure focus on effective flow restriction with the add-on hemolysis-reduction accessory (also referred to herein as a flow restriction device) that regulates the overall flow rate of the entire fluid path as blood cells travel through. The flow restriction device can be either assembled with the PIVC or co-packaged with the PIVC. As such, there is no additional operation during catheter placement since the device has a vented lumen that enables blood flashback. The clinician may connect a blood collection device to the port of the accessory and can then draw blood to the intended volume. After blood draw, the clinician may disconnect and discard the flow restriction device and the blood collection device together. As such, this flow restriction device can be either for single blood draw or stay inline throughout indwell.

The flow restriction devices and associated blood collection systems of the various embodiments described herein additionally provide further advantages over currently existing blood collection systems. For example, add-on flow restriction devices described herein allow for hemolysis-reduction function to be integrated for PIVC blood draw. Further, the flow restriction devices described herein are compatible with PIVC placement and allow for seamless blood draw at insertion. Additionally, since the flow restriction devices are an add-on which can be easily incorporated without any changes to existing PIVC, there is minimal impact to clinical setting and operations.

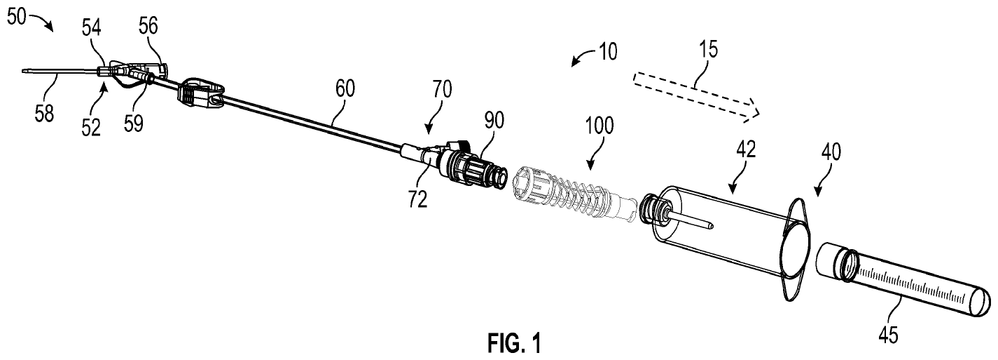

FIG. 1 illustrates a vascular access device 10 including a peripheral intravenous catheter (PIVC) assembly 50 that includes a flow restriction device 100, in accordance with some embodiments of the present disclosure. The flow restriction device 100 may be configured to reduce a likelihood of hemolysis during blood collection using the vascular access device 10. In some embodiments, the vascular access device 10 may include a catheter assembly 50. The catheter assembly 50 may include a catheter hub 52, which may include a distal end 54, a proximal end 56, and a lumen extending through the distal end and the proximal end. The catheter assembly 50 may further include a catheter 58, which may be secured within the catheter hub 52 and may extend distally from the distal end 54 of the catheter hub 52. In some embodiments, the catheter assembly 50 may be a peripheral intravenous catheter (PIVC).

In some embodiments, the catheter assembly 50 may include or correspond to any suitable catheter assembly 50. In some embodiments, the catheter assembly 50 may be integrated and include an extension tube 60, which may extend from and be integrated with a side port 59 of the catheter hub 52. A non-limiting example of an integrated catheter assembly is the BD NEXIVA™ Closed IV Catheter system, available from Becton Dickinson and Company. In some embodiments, a proximal end of the extension tube 60 may be coupled to an adapter, such as, for example, a Y-adapter 70. In some embodiments, the flow restriction device 110 may be fluidly coupled to the Y-adapter 70.

In some embodiments, the catheter assembly 50 may be non-integrated and may not include the extension tube 60. In these and other embodiments, the flow restriction device 100 may be configured to couple to the proximal end 56 of the catheter hub 52 or another suitable portion of the catheter assembly 50. In some embodiments, the flow restriction device 100 may be coupled directly to the catheter assembly 50, eliminating the extension tube 60 and providing a compact catheter system.

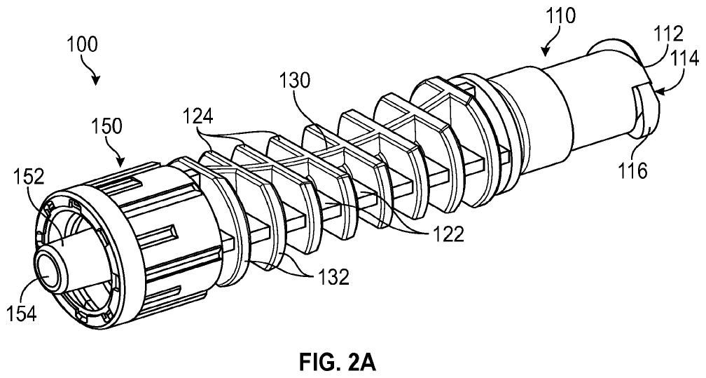

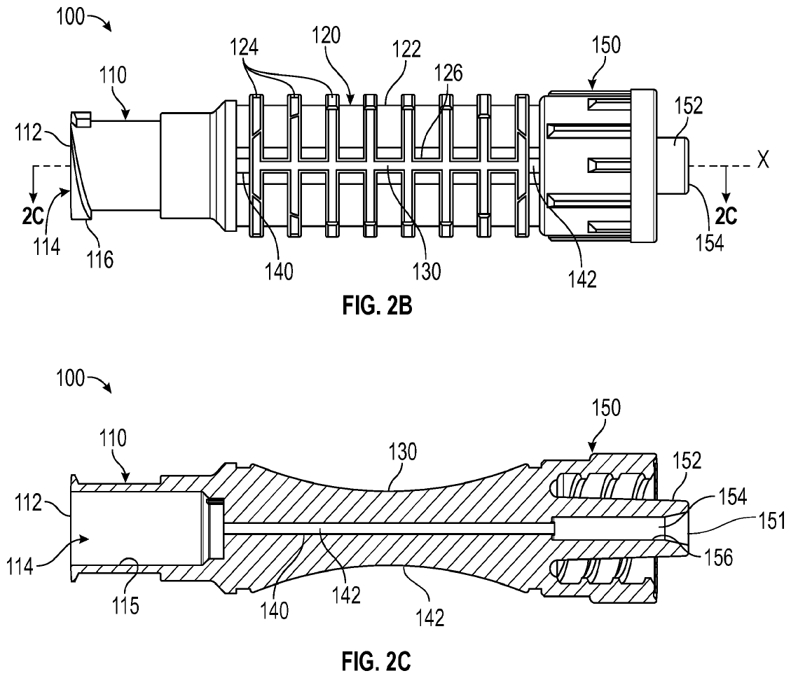

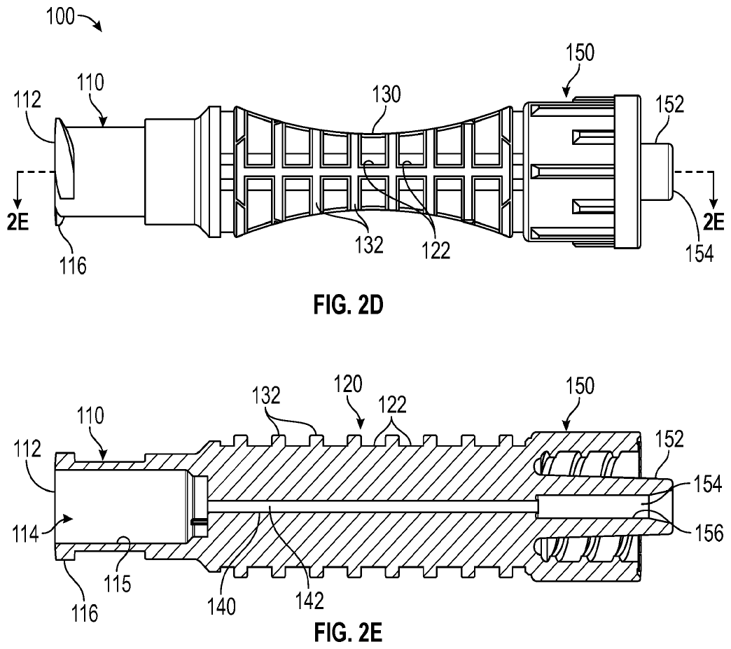

FIG. 2A illustrates a perspective view of the flow restriction device, in accordance with some embodiments of the present disclosure. FIG. 2B illustrates a top view of the flow restriction device of FIG. 2A, in accordance with some embodiments of the present disclosure. FIG. 2C illustrates a cross-sectional view of the flow restriction device of FIG. 2B along line 2C-2C, in accordance with some embodiments of the present disclosure. FIG. 2D illustrates a side view of the flow restriction device of FIG. 2A, in accordance with some embodiments of the present disclosure. FIG. 2E illustrates a cross-sectional view of the flow restriction device of FIG. 2D along the line 2E-2E, in accordance with some embodiments of the present disclosure.

As illustrated in FIGS. 2A-2E, with continued reference to FIG. 1, in some embodiments, the flow restriction device 100 may include a male luer connector portion 150 configured to couple to the catheter assembly 50. The male luer connector portion 150 may have an internal surface 156 defining a lumen 154 of the male luer connector portion 150. In some embodiments, the flow restriction device 100 may further include a female luer connector portion 110 disposed proximally to the male luer connector portion 150. The female luer connector portion 110 may be configured to couple to fluid collection device 40 (e.g., a blood collection device). For example, the female luer connector portion 110 may be integrated with the blood collection device 40 or monolithically formed with the blood collection device 40 as a single unit or piece. As another example, the female luer connector portion 110 may be in the form of a female luer connector or another suitable connector, which may be coupled with a male luer portion of the blood collection device 40. The female luer connector portion 110 may include an internal surface 115 defining a lumen 114 extending therethrough for coupling to the male luer portion of the blood collection device 40. The lumen of the 114 of the female luer connector portion 110 may be fluidly connected to the lumen 154 of the male luer connector portion via a recess or lumen 140 of a body portion 120 of the flow restriction device 100, as shall be described below.

In accordance with various embodiments of the present disclosure, the flow restriction device 100 may further include the body portion 120 extending between the male luer 150 portion and the female luer portion 110, and integrally formed with the male luer portion 150 and the female luer portion 110 to form a single monolithic piece. The aforementioned configuration in which the flow restriction device 100 is formed as a single monolithic piece is advantageous over currently existing blood draw connectors which generally include a plurality of connectors coupled to each other. For example, the structure of the flow restriction device 100 being formed as a single monolithic piece advantageously provides a design of reduced complexity and reduced cost as compared to currently existing blood draw connectors which incorporate several parts and connectors. The structure of the flow restriction device 100 being formed as a single monolithic piece also advantageously reduces wastage of material as compared to currently existing blood draw connectors which incorporate several parts and connectors. Further advantageously, the structure of the flow restriction device 100 being formed as a single monolithic piece reduces or otherwise eliminates the need for performing leakage testing, thereby saving time and cost associated with the testing.

As depicted, the body portion 120 may include a recess or lumen 140 extending longitudinally therethrough and fluidly communicated with the lumens 154 and 114 of the male luer portion 150 and the female luer portion 110. The lumen or recess 140 may defines a micro-channel 142 along which a fluid flows from the male luer connector portion 150 into the fluid collection device 40 via the female luer connector portion 110. As depicted, the lumen or recess 140 may fluidly communicate the catheter assembly 50 with the fluid collection device 40 via the flow restriction device 100. For example, in some embodiments, a leg 72 of the Y-adapter 70 may be coupled to the flow restriction device 100. The leg 72 of Y-adapter 70 may include a lumen into which the distal end 151 of the male luer connector portion 150 may be coupled. The Y-adapter 70 may fluidly communicate the flow restriction device 100 via a connector 90, which is depicted as a needleless connector, and recess or lumen 140 with the catheter assembly 50, for example, via the extension tubing 60. Accordingly, the lumen or recess 140 of the micro-channel 142 may define a fluid pathway with a reduced, small, or micro-sized diameter (as discussed below) through which fluid entering the flow restriction device 100 from the catheter assembly 50, may flow through the flow restriction device 100 for collection in the fluid collection device 40. For example, where blood is being withdrawn or collected from a patient, the medical fluid may be blood, and the fluid collection device 40 may be a blood collection device. In some embodiments, the blood collection device may be a luer lock access device (LLAD). Accordingly, during blood collection or withdrawal from the patients, the blood sample may flow from the distal end 151 of the male luer connector portion 150 into the LLAD 40 via the flowpath or micro-channel 142 defined by the recess or lumen 140.

In some embodiments, micro-channel 142 defined by the recess or lumen 140 may be an elongate, thin channel having a small, reduced, or micro-sized diameter. For example, in some embodiments, the recess or lumen 140 which defines the flowpath or micro-channel along which fluid may flow from a distal end 151 into the fluid collection device 40, may have a diameter in the range of a twenty thousandth to twenty-five thousandth of an inch. However, the various embodiments of the present disclosure are not limited to the aforementioned configuration. In some embodiments, the diameter of the recess or lumen 142 of the defining the micro-channel may range from 1 to 1.3 inches. Accordingly, during blood collection or withdrawal from the patient, the blood 15 may flow into the blood collection device 40 via the flowpath or micro-channel defined by the recess or lumen 142 having minimal diameter. The flow restriction device 100 of the various embodiments described herein is advantageous over currently existing blood collection systems. For example, during blood draw with currently existing blood draw devices, blood cells may experience wall shear stress as they flow from the distal end to the proximal end of the blood collection systems. Wall shear stress on blood cells is considered a major source of mechanical damage to blood cells causing hemolysis of the blood cells. The flowpath or micro-channel defined by the lumen 142 having minimal diameter may facilitate increased flow resistance within the vascular access system to distribute the pressure differential and reduce shear stress experienced by the red blood cells of the blood 15. For example, the minimized diameter of the flowpath or micro-channel defined by the lumen 142 may provide increased resistance to flow of the blood 15 and thereby decrease blood flow rate within the flow resistance device 100. Since the decreased blood flow rate causes a reduction in shear stress experienced by the red blood cells of the blood 15, a risk of hemolysis during blood collection may advantageously be reduced.

The flow restriction device 100 of the various embodiments described herein having the integrated flowpath or micro-channel defined by the lumen 142 of the recess 140 may be advantageous in cost over currently existing blood draw connectors or accessories which incorporate a cannula which tends to increase the cost of the blood draw connectors or accessories significantly.

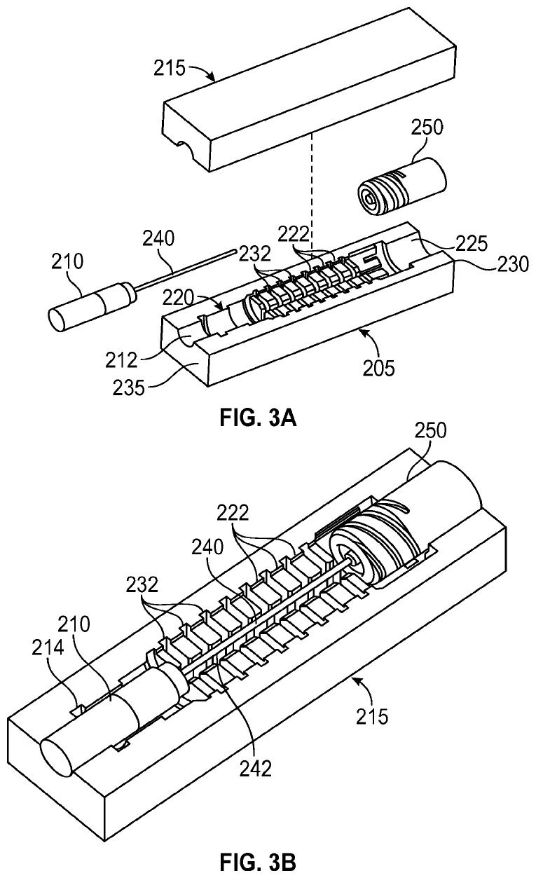

According to various embodiments of the present disclosure, an outer surface 122 of the body portion 120 may include a plurality of laterally extending ribs 124 disposed about and surrounding a longitudinal axis X of the body portion 120. As depicted, the plurality of laterally extending ribs 124 may be spaced apart from each other along the longitudinal axis X between the female luer connector portion 110 and the male luer connector portion 150. In some embodiments, the outer surface 122 may further include at least one longitudinally extending rib 130 disposed along the longitudinal axis X and extending from the male luer connector portion 150 to the female luer portion 110. In some embodiments, as illustrated in FIG. 3, the body portion 120 may include a pair of longitudinally extending ribs 130 disposed at opposing sides of the body portion 120. As depicted, the longitudinally extending rib 130 may be disposed transverse to and may interconnect each of the laterally extending ribs 124. The plurality of laterally extending ribs 124 and the one or more longitudinally extending ribs 130 may thus form a grid or matrix shape surrounding the body portion 120.

The grid or matrix shape surrounding the body portion 120 may advantageously provide additional or enhanced structural integrity or rigidity as compared with currently existing connectors for blood collection systems. For example, in some embodiments, the grid or matrix shape of the laterally extending ribs 124 and the longitudinally extending ribs 130 surrounding the body portion 120 may provide increased rigidity between the male luer connector portion 150 and the female luer connector portion 110 of the flow restriction device 100, thereby making the flow restriction device 100 less susceptible to bending or twisting forces. The aforementioned configuration is advantageous in that the enhanced rigidity of the flow restriction device 100 makes it less flexible thereby decreasing the possibility of blood spillage due to accidental or inadvertent bending or twisting of the flow control device 100. The aforementioned configuration of the flow restriction device with the laterally extending ribs 124 and the longitudinally extending ribs 130 surrounding the body portion 120 may further be advantageous in providing a surface on the body portion that is easier to grip than would a more uniform surface of the body portion 120 be to grip.

FIG. 3A illustrates perspective view of a plurality of mold parts for forming a flow restriction device, in accordance with some embodiments of the present disclosure. FIG. 3B illustrates an assembled mold of the flow restriction device without a top mold of the flow restriction device, in accordance with some embodiments of the present disclosure. In accordance with some embodiments, a method of manufacturing a single monolithic piece flow restriction device 100 may include providing first and second mold parts 205 and 215. In some embodiments, the first and second mold parts 205 and 215 may be identical in structure and mirror each other. As depicted, each of the first and second mold parts 205 and 215 may include an imprint 220 recessed therein. The imprint 220 may include a male luer connector imprint portion 225 at a distal end portion 230 of the first mold part 205, a female luer connector imprint portion 212 at a proximal end portion thereof 235, a micro-channel imprint portion 242 extending longitudinally between the male luer connector imprint portion 225 and the female luer connector imprint portion 212, and a reinforcing rib imprint portion 222 and 232 surrounding the micro-channel imprint portion 242.

In some embodiments, the method of manufacturing a single monolithic piece flow restriction device 100 may further include inserting a male luer connector mold part 250 into the male luer connector imprint portion 225, and inserting a female luer connector mold part 210 into the female luer connector imprint portion 212. As depicted in FIG. 3B, the female luer connector mold part 210 may have a micro-channel mold part 240 extending from a distal end of the female luer connector mold part 210, and the micro-channel mold part may be inserted into the micro-channel imprint portion 242. According to various embodiments of the present disclosure the method may further include coupling the first and second mold parts 205 and 215 to form a mold assembly, and injecting a molten mold material into the mold assembly. The method may further include cooling the mold assembly to allow the molten mold material to cool into the shape of the flow restriction device 100.

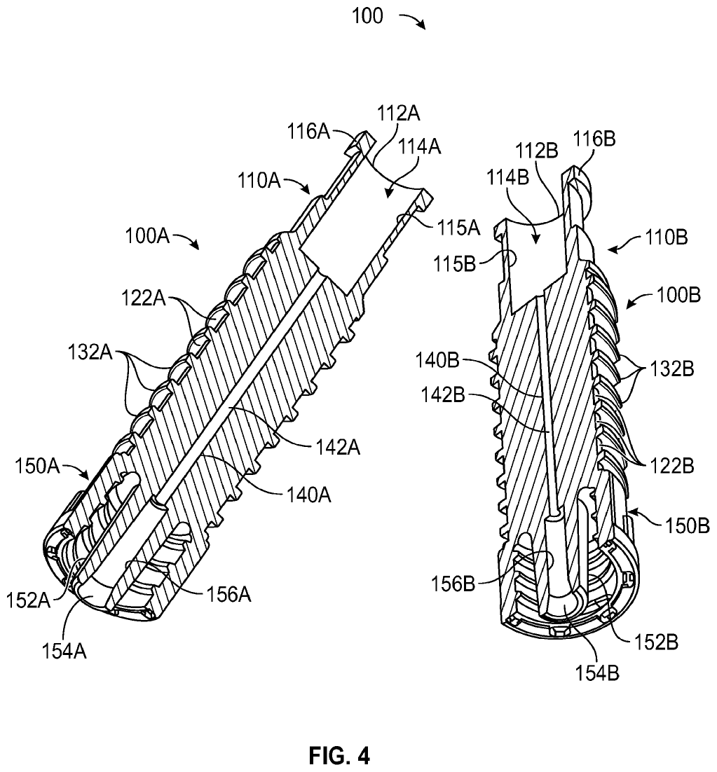

FIG. 4 illustrates a cross-sectional view of first and second connector halves 100A and 100B of a flow restriction device 100, in accordance with some embodiments of the present disclosure. According to various embodiments of the present disclosure, the single piece flow restriction device 100 may be formed by permanently bonding two identical halves 100A and 100B, as illustrated in FIG. 4. The structure of the two connector halves 100A and 100B when combined may be identical to the structure of the flow restriction device 100. Accordingly, the same reference characters have been used to denote corresponding features in the first and second connector halves 100A and 100B, with the exception that the corresponding elements in the first and second connector halves 100A and 100B include either an “A” or a “B” at the end of the reference character to denote the respective connector half to which the feature corresponds. Accordingly, a detailed description of the features of the first and second connector halves 100A and 100B which are identical to those of the flow restriction device 100 are omitted with respect to FIG. 4. For example, in some embodiments, the single piece flow restriction device 100 may be formed by ultrasonically welding the two halves 100A and 100B together. In particular, in some embodiments, the first and second halves 100A and 100B may be ultrasonically welded about a weld line defined along the internal surface 156A, 156B defining the lumen 154A, 154B of the male luer connector portion 150A, 150B, the internal surface 115A, 115B defining the lumen 114A, 114B of the female luer connector portion 110A, 110B, and an internal surface 156A, 156B of the at least one recess or lumen 140A, 140B defining the micro-channel 142A, 142B. However, the various embodiments of the present disclosure are not limited to the aforementioned configuration. In some embodiments, the first and second connector halves 100A, 100B may be permanently bonded to each other by any other appropriate boding or attachment method.

The aforementioned configuration of the flow restriction device 100 having the first and second connector halves 100A and 100B may be advantageous in that the weld line defined along the internal surface 156A, 156B defining the lumen 154A, 154B of the male luer connector portion 152A, 152B, the internal surface 115A, 115B defining the lumen 114A, 114B of the female luer connector portion 110A, 110B, and an internal surface 156A, 156B of the at least one recess or lumen 140A, 140B defining the micro-channel 142A, 142B may provide additional or enhanced sealing of the fluid path from the male luer connector portion 150 to the female luer connector portion 110 so as to prevent leakage of the medical fluid, for example, blood during blood draw.

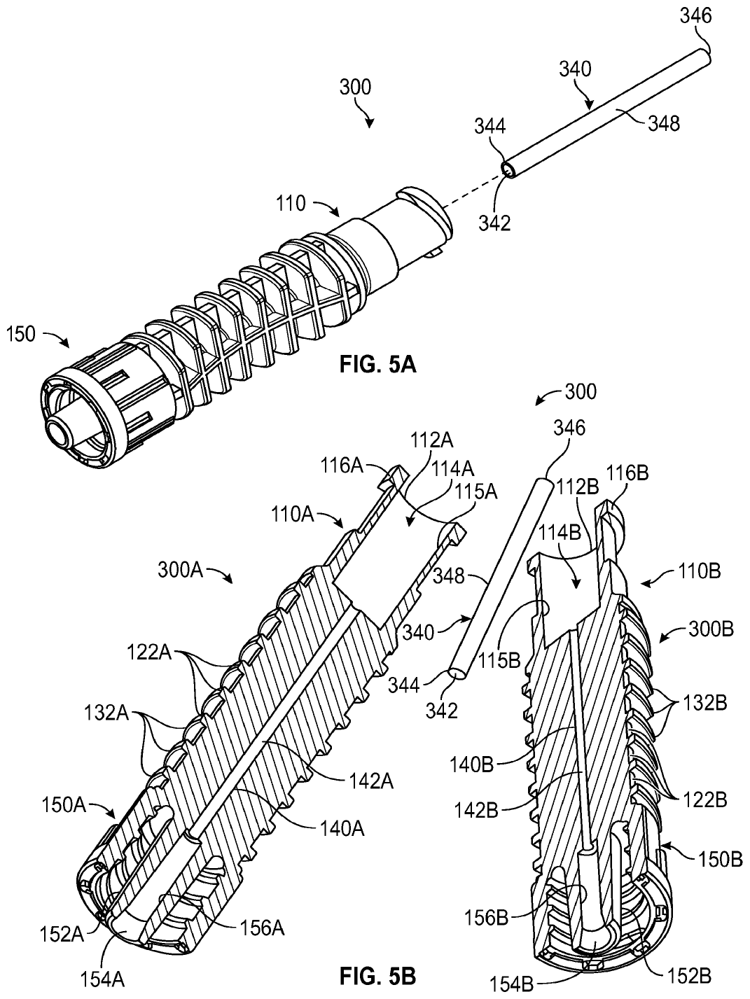

FIG. 5A illustrates a perspective view of a flow restriction device 300, in accordance with some embodiments of the present disclosure. FIG. 5B illustrates a cross-sectional view of first and second halves 300A, 300B of the flow restriction device of FIG. 5A, in accordance with some embodiments of the present disclosure. As depicted, the structure of the two connector halves 300A and 300B may be identical to the structure of the two connector halves 100A and 100B flow restriction device 100, with the exception that the flow restriction device 300 may further include a tubing 340 to be mounted in the lumen or recess 140 (i.e., 140A and 140B). Accordingly, the same reference characters have been used to denote corresponding features in the first and second connector halves 300A and 300B. As such, a detailed description of the features of the first and second connector halves 300A and 300B which are identical to those of the flow restriction device 100 are omitted with respect to FIGS. 5A and 5B. For example, in some embodiments, the single piece flow restriction device 300 may be formed by ultrasonically welding the two halves 300A and 300B together with the tubing 340 sandwiched therebetween.

In some embodiments, tubing 340 may have a proximal end 346, a distal end 344, an inner surface defining a lumen 342 extending therethrough. The tubing 340 may be mounted in the lumen or recess 140 and may be interposed between and extend between the lumens 114 and 154 of the female luer connector portion 110 and the male luer connector portion. In accordance with some embodiments of the present disclosure, the lumen 342 of the tubing 340 may define a flowpath or micro-channel along which a fluid (e.g., blood) may flow from the flow restriction device 300 into the fluid collection device 40. In some embodiments, the flowpath or micro-channel defined by the lumen 342 of the tubing may be an elongate, thin channel having a small, reduced, or micro-sized diameter. For example, in some embodiments, the flowpath or micro-channel defined by the lumen 342 may have a diameter in the range of a twenty thousandth to twenty-five thousandth of an inch. In some embodiments, the tubing 340 may be removed following the joining of the two halves 300A and 300B, and during the joining of the two halves, with the tubing therebetween, the tubing can contribute to the proper alignment of the micro-channel 142A, 142B. However, the various embodiments of the present disclosure are not limited to the aforementioned configurations.

In some embodiments, the diameter of the flowpath or micro-channel defined by the lumen 342 may range from 1 to 1.3 inches. Accordingly, during blood collection or withdrawal from the patient, the blood may flow into the blood collection device 40 via the flowpath or micro-channel defined by the lumen 342 having minimal diameter. The flowpath or micro-channel defined by the lumen 342 of tubing 340 having minimal diameter may facilitate increased flow resistance within the vascular access system to distribute the pressure differential and reduce shear stress experienced by the red blood cells of the blood. For example, the minimized diameter of the flowpath or micro-channel defined by the lumen 342 may provide increased resistance to flow of the blood and thereby decrease blood flow rate within the flow restriction device 300. Since the decreased blood flow rate causes a reduction in shear stress experienced by the red blood cells of the blood 15, a risk of hemolysis during blood collection may advantageously be reduced.

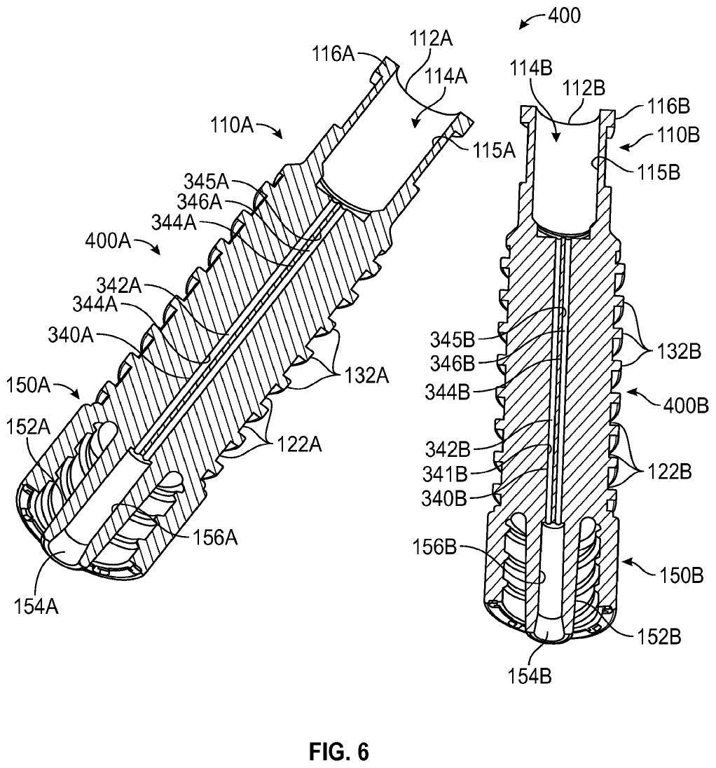

FIG. 6 illustrates a cross-sectional view of first and second connector halves 400A and 400B of a flow restriction device 400, in accordance with some embodiments of the present disclosure. As depicted, the structure of the two connector halves 400A and 400B may be identical to the structure of the two connector halves 100A and 100B of flow restriction device 100, with the exception that the flow restriction device 400 may include first and second lumens or recesses 440A, 440B and 444A, 444B. Accordingly, the same reference characters have been used to denote corresponding features in the first and second connector halves 400A and 400B. As such, a detailed description of the features of the first and second connector halves 400A and 400B which are identical to those of the flow restriction device 100 are omitted with respect to FIG. 6. For example, in some embodiments, the single piece flow restriction device 400 may be formed by ultrasonically welding the two halves 400A and 400B together.

In some embodiments, the first lumen or recess 440 defined by the combined lumen or recess halves 440A, 440B may have an internal surface 441A, 441B defining a micro-channel 442A, 442B therein. Similarly, the second lumen or recess 444 defined by the combined lumen or recess halves 444A, 444B may have an internal surface 445A, 445B defining a micro-channel 446A, 446B therein. According to various embodiments of the present disclosure, the flowpath defined in each of the micro-channels 442A, 442B, 446A, and 446B may be an elongate, thin channel having a small, reduced, or micro-sized diameter. For example, in some embodiments, the flowpath defined in each of the micro-channels 442A, 442B, 446A, and 446B may have a diameter in the range of a twenty thousandth to twenty-five thousandth of an inch. However, the various embodiments of the present disclosure are not limited to the aforementioned configuration. In some embodiments, the diameter of the flowpath or micro-channel defined by the lumen 442 may range from 1 to 1.3 inches. Accordingly, during blood collection or withdrawal from the patient, the blood may flow into the blood collection device 40 via the flowpath or micro-channel defined by the lumen 442 having minimal diameter.

The flowpath defined in each of the micro-channels 442A, 442B, 446A, and 446B having minimal diameter may advantageously facilitate increased flow resistance within the vascular access system to distribute the pressure differential and reduce shear stress experienced by the red blood cells of the blood being drawn. For example, the minimized diameter of the flowpath defined in each of the micro-channels 442A, 442B, 446A, and 446B may provide increased resistance to flow of the blood 15 and thereby decrease blood flow rate within the flow resistance device 400. Since the decreased blood flow rate causes a reduction in shear stress experienced by the red blood cells of the blood, a risk of hemolysis during blood collection may advantageously be reduced.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A flow restriction device, comprising: a male luer connector portion configured to couple to a catheter assembly, the male luer connector portion comprising an internal surface defining a lumen thereof; a female luer connector portion disposed proximally to the male luer connector portion and configured to couple to a fluid collection device, the female luer connector portion comprising an internal surface defining a lumen fluidly connected to the lumen of the male luer connector portion; and a body portion extending between the male luer portion and the female luer portion, and integrally formed with the male luer portion and the female luer portion to form a single monolithic piece, the body portion comprising a recess extending longitudinally therethrough and fluidly communicated with the lumens of the male luer portion and the female luer portion, wherein the recess defines a micro-channel along which a fluid flows from the male luer connector portion into the fluid collection device via the female luer connector portion.

Clause 2. The flow restriction device of Clause 1, wherein an outer surface of the body portion comprises a plurality of laterally extending ribs disposed about and surrounding a longitudinal axis of the body portion, the plurality of ribs being spaced apart from each other along the longitudinal axis between the female luer connector portion and the male luer connector portion.

Clause 3. The flow restriction device of Clause 2, wherein the outer surface of the body portion further comprises at least one longitudinally extending rib disposed along the longitudinal axis and extending from the male luer connector portion to the female luer connector portion.

Clause 4. The flow restriction device of Clause 3, wherein the longitudinally extending rib is disposed transverse to and interconnects each of the laterally extending ribs

Clause 5. The flow restriction device of Clause 3, wherein the least one longitudinally extending rib comprises a pair of longitudinally extending ribs disposed on opposing sides of the outer surface of the body portion.

Clause 6. The flow restriction device of Clause 5, wherein the pair of longitudinally extending ribs are disposed transverse to and interconnect each of the laterally extending ribs on opposing sides of the outer surface of the body portion.

Clause 7. The flow restriction device of Clause 4, wherein the fluid flowing from the male luer connector portion into the fluid collection device via the female luer connector portion comprises blood and the fluid collection device comprises a blood collection device.

Clause 8. The flow restriction device of Clause 8, wherein the blood collection device comprises a luer lock access device.

Clause 9. A method of manufacturing a single monolithic piece flow restriction device configured to limit hemolysis during the drawing of blood from a patient, the method comprising: providing first and second mold parts, each of the first and second mold parts comprising an imprint recessed therein, the imprint comprising a male luer connector imprint portion at a distal end portion of the first mold part, a female luer connector imprint portion at a proximal end portion thereof, a micro-channel imprint portion extending longitudinally between the male luer connector imprint portion and the female luer connector imprint portion, and a reinforcing rib imprint portion surrounding the micro-channel imprint portion; inserting a male luer connector mold part into the male luer connector imprint portion; inserting a female luer connector mold part into the female luer connector imprint portion, the female luer connector mold part comprising a micro-channel mold part extending from a distal end of the female luer connector mold part, wherein the micro-channel mold part is inserted into the micro-channel imprint portion; coupling the first and second mold parts to form a mold assembly; injecting a molten mold material into the mold assembly; and cooling the mold assembly.

Clause 10. A flow restriction device, comprising: first and second connector halves fused together to form a single connector, the single connector comprising: a male luer connector portion configured to couple to a catheter assembly, the male luer connector portion comprising an internal surface defining a lumen thereof; a female luer connector portion disposed proximally to the male luer connector portion and configured to couple to a fluid collection device, the female luer connector portion comprising an internal surface defining a lumen fluidly connected to the lumen of the male luer connector portion; and a body portion extending between the male luer portion and the female luer portion, and integrally formed with the male luer portion and the female luer portion to form a single monolithic piece, the body portion comprising at least one recess extending longitudinally therethrough and fluidly communicated with the lumens of the male luer portion and the female luer portion, wherein the at least one recess defines a micro-channel along which a fluid flows from the male luer connector portion into the fluid collection device via the female luer connector portion.

Clause 11. The flow restriction device of Clause 10, wherein the first and second connector halves are ultrasonically welded together about a weld line defined along the internal surface defining the lumen of the male luer connector portion, the internal surface defining the lumen of the female luer connector portion, and an internal surface of the at least one recess defining the micro-channel.

Clause 12. The flow restriction device of Clause 11, wherein the lumen of the male luer connector portion, the micro-channel, and the lumen of the female luer connector portion define a fluid pathway along which fluid travels through the flow restriction device, and the weld line seals an outer periphery of the fluid pathway from leakage.

Clause 13. The flow restriction device of Clause 11, wherein an outer surface of the body portion comprises a plurality of laterally extending ribs disposed about and surrounding a longitudinal axis of the body portion, the plurality of ribs being spaced apart from each other along the longitudinal axis between the female luer connector portion and the male luer connector portion.

Clause 14. The flow restriction device of Clause 13, wherein the outer surface of the body portion further comprises at least one longitudinally extending rib disposed along the longitudinal axis and extending from the male luer portion to the female luer portion.

Clause 15. The flow restriction device of Clause 14, wherein the at least one longitudinally extending rib is disposed transverse to and interconnects each of the laterally extending ribs.

Clause 16. The flow restriction device of Clause 13, wherein the least one longitudinally extending rib comprises a pair of longitudinally extending ribs each disposed on a corresponding half of sides of the outer surface of the body portion.

Clause 17. The flow restriction device of Clause 11, further comprising a tubing mounted in the recess defining the micro-channel.

Clause 18. The flow restriction device of Clause 10, wherein the at least one recess comprises a plurality of recesses defining a plurality of micro-channels each fluidly communicating the lumen of the male luer connector portion with the lumen of the female luer connector portion.

Clause 19. The flow restriction device of Clause 18, wherein the first and second connector halves are ultrasonically welded together about a plurality of weld lines, each defined along the internal surface defining the lumen of the male luer connector portion, the internal surface defining the lumen of the female luer connector portion, and each of a plurality of internal surfaces of the at least one recess defining the micro-channel.

Clause 20. The flow restriction device of Clause 19, wherein the lumen of the male luer connector portion, each of the plurality of micro-channels, and the lumen of the female luer connector portion define a plurality of fluid pathways along which fluid travels through the flow restriction device, and the weld lines seals an outer periphery of each fluid pathway from leakage.

Clause 21. A peripheral intravenous catheter assembly configured to limit hemolysis during the drawing of blood from a patient, comprising: a flow restriction device, comprising, a male luer connector portion configured to couple to a catheter assembly, the male luer connector portion comprising an internal surface defining a lumen thereof; a female luer connector portion disposed proximally to the male luer connector portion and configured to couple to a fluid collection device, the female luer connector portion comprising an internal surface defining a lumen fluidly connected to the lumen of the male luer connector portion; and a body portion extending between the male luer portion and the female luer portion, and integrally formed with the male luer portion and the female luer portion to form a single monolithic piece, the body portion comprising a recess extending longitudinally therethrough and fluidly communicated with the lumens of the male luer portion and the female luer portion, wherein the recess defines a micro-channel, the micro-channel configured to limit hemolysis of blood as the blood is drawn from a patient from the male luer connector portion into the fluid collection device via the female luer connector portion; a catheter hub having a proximal end and a distal end; and a fluid connector that fluidly couples the catheter hub with the flow restriction device.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. The accompanying method claims present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

1. A flow restriction device, comprising:
a male luer connector portion configured to couple to a catheter assembly, the male luer connector portion comprising an internal surface defining a lumen thereof;
a female luer connector portion disposed proximally to the male luer connector portion and configured to couple to a fluid collection device, the female luer connector portion comprising an internal surface defining a lumen fluidly connected to the lumen of the male luer connector portion; and
a body portion extending between the male luer portion and the female luer portion, and integrally formed with the male luer portion and the female luer portion to form a single monolithic piece, the body portion comprising a recess extending longitudinally therethrough and fluidly communicated with the lumens of the male luer portion and the female luer portion, wherein the recess defines a micro-channel along which a fluid flows from the male luer connector portion into the fluid collection device via the female luer connector portion.

2. The flow restriction device of claim 1, wherein an outer surface of the body portion comprises a plurality of laterally extending ribs disposed about and surrounding a longitudinal axis of the body portion, the plurality of ribs being spaced apart from each other along the longitudinal axis between the female luer connector portion and the male luer connector portion.

3. The flow restriction device of claim 2, wherein the outer surface of the body portion further comprises at least one longitudinally extending rib disposed along the longitudinal axis and extending from the male luer connector portion to the female luer connector portion.

4. The flow restriction device of claim 3, wherein the longitudinally extending rib is disposed transverse to and interconnects each of the laterally extending ribs.

5. The flow restriction device of claim 3, wherein the least one longitudinally extending rib comprises a pair of longitudinally extending ribs disposed on opposing sides of the outer surface of the body portion.

6. The flow restriction device of claim 5, wherein the pair of longitudinally extending ribs are disposed transverse to and interconnect each of the laterally extending ribs on opposing sides of the outer surface of the body portion.

7. The flow restriction device of claim 4, wherein the fluid flowing from the male luer connector portion into the fluid collection device via the female luer connector portion comprises blood and the fluid collection device comprises a blood collection device.

8. The flow restriction device of claim 8, wherein the blood collection device comprises a luer lock access device.

9. A method of manufacturing a single monolithic piece flow restriction device configured to limit hemolysis during the drawing of blood from a patient, the method comprising:
providing first and second mold parts, each of the first and second mold parts comprising an imprint recessed therein, the imprint comprising a male luer connector imprint portion at a distal end portion of the first mold part, a female luer connector imprint portion at a proximal end portion thereof, a micro-channel imprint portion extending longitudinally between the male luer connector imprint portion and the female luer connector imprint portion, and a reinforcing rib imprint portion surrounding the micro-channel imprint portion;
inserting a male luer connector mold part into the male luer connector imprint portion;
inserting a female luer connector mold part into the female luer connector imprint portion, the female luer connector mold part comprising a micro-channel mold part extending from a distal end of the female luer connector mold part, wherein the micro-channel mold part is inserted into the micro-channel imprint portion;
coupling the first and second mold parts to form a mold assembly;
injecting a molten mold material into the mold assembly; and
cooling the mold assembly.

10. A flow restriction device, comprising:
first and second connector halves fused together to form a single connector, the single connector comprising:
a male luer connector portion configured to couple to a catheter assembly, the male luer connector portion comprising an internal surface defining a lumen thereof;
a female luer connector portion disposed proximally to the male luer connector portion and configured to couple to a fluid collection device, the female luer connector portion comprising an internal surface defining a lumen fluidly connected to the lumen of the male luer connector portion; and
a body portion extending between the male luer portion and the female luer portion, and integrally formed with the male luer portion and the female luer portion to form a single monolithic piece, the body portion comprising at least one recess extending longitudinally therethrough and fluidly communicated with the lumens of the male luer portion and the female luer portion, wherein the at least one recess defines a micro-channel along which a fluid flows from the male luer connector portion into the fluid collection device via the female luer connector portion.

11. The flow restriction device of claim 10, wherein the first and second connector halves are ultrasonically welded together about a weld line defined along the internal surface defining the lumen of the male luer connector portion, the internal surface defining the lumen of the female luer connector portion, and an internal surface of the at least one recess defining the micro-channel.

12. The flow restriction device of claim 11, wherein the lumen of the male luer connector portion, the micro-channel, and the lumen of the female luer connector portion define a fluid pathway along which fluid travels through the flow restriction device, and the weld line seals an outer periphery of the fluid pathway from leakage.

13. The flow restriction device of claim 11, wherein an outer surface of the body portion comprises a plurality of laterally extending ribs disposed about and surrounding a longitudinal axis of the body portion, the plurality of ribs being spaced apart from each other along the longitudinal axis between the female luer connector portion and the male luer connector portion.

14. The flow restriction device of claim 13, wherein the outer surface of the body portion further comprises at least one longitudinally extending rib disposed along the longitudinal axis and extending from the male luer portion to the female luer portion.

15. The flow restriction device of claim 14, wherein the at least one longitudinally extending rib is disposed transverse to and interconnects each of the laterally extending ribs.

16. The flow restriction device of claim 13, wherein the least one longitudinally extending rib comprises a pair of longitudinally extending ribs each disposed on a corresponding half of sides of the outer surface of the body portion.

17. The flow restriction device of claim 11, further comprising a tubing mounted in the recess defining the micro-channel.

18. The flow restriction device of claim 10, wherein the at least one recess comprises a plurality of recesses defining a plurality of micro-channels each fluidly communicating the lumen of the male luer connector portion with the lumen of the female luer connector portion.

19. The flow restriction device of claim 18, wherein the first and second connector halves are ultrasonically welded together about a plurality of weld lines, each defined along the internal surface defining the lumen of the male luer connector portion, the internal surface defining the lumen of the female luer connector portion, and each of a plurality of internal surfaces of the at least one recess defining the micro-channel.

20. A peripheral intravenous catheter assembly configured to limit hemolysis during the drawing of blood from a patient, comprising:
a flow restriction device, comprising,
a male luer connector portion configured to couple to a catheter assembly, the male luer connector portion comprising an internal surface defining a lumen thereof;
a female luer connector portion disposed proximally to the male luer connector portion and configured to couple to a fluid collection device, the female luer connector portion comprising an internal surface defining a lumen fluidly connected to the lumen of the male luer connector portion; and
a body portion extending between the male luer portion and the female luer portion, and integrally formed with the male luer portion and the female luer portion to form a single monolithic piece, the body portion comprising a recess extending longitudinally therethrough and fluidly communicated with the lumens of the male luer portion and the female luer portion, wherein the recess defines a micro-channel, the micro-channel configured to limit hemolysis of blood as the blood is drawn from a patient from the male luer connector portion into the fluid collection device via the female luer connector portion;
a catheter hub having a proximal end and a distal end; and
a fluid connector that fluidly couples the catheter hub with the flow restriction device.
